(19) United States
(12) Patent Application Publication
(10) Pub. No.: US 2008/0221367 A1
(43) Pub. Date: Sep. 11, 2008

(54) Horizontal Reactor Vessel

(76) Inventors: Elco Dick Hollander, Amsterdam (NL); Peter Anton August Klusener, Amsterdam (NL); Ingmar Hubertus Josephina Ploemen, Moerdijk (NL); Cornelius Johannes Schellekens, Amsterdam (NL)

Correspondence Address: SHELL OIL COMPANY, P O BOX 2463, HOUSTON, TX, 772522463, US

(21) Appl. No.: 11/661,346

(22) PCT Filed: Sep. 1, 2005
(86) PCT No.: PCT/EP2005/054305

§ 371 Date: May 13, 2008

(30) Foreign Application Priority Data
Sep. 1, 2004 (EP) 04255273.7

(51) Int. Cl. C07C 409/02; B01J 19/00; C07C 13/16

(52) U.S. Cl. 568/571; 422/129; 422/198; 422/188; 585/24

(57) ABSTRACT

Horizontal reactor vessel (1) having a lower part (3) and two opposite ends (9, 10), which reactor vessel comprises a liquid inlet (13) at one end (9), a fluid outlet (14) at the opposite end (10) and a gas inlet device (17) arranged in the lower part (3), which reactor vessel contains at least one substantially vertical baffle-plate (23) arranged in the direction of liquid flow through the reactor vessel (1) during normal operation.

The present invention relates to a horizontal reactor vessel, especially a horizontal reactor vessel for contacting a liquid reactant, such as ethylbenzene or cumene, with a gaseous reactant, such as oxygen in order to obtain an organic hydroperoxide.

Horizontal reactor vessels are known in the art and have been described for example in U.S. Pat. No. 4,269,805.

There is still room for improving horizontal reactor vessels for contacting gaseous and liquid reactant. A better contact between the gaseous and the liquid reactants generally is desirable as this tends to make the reaction of the liquid reactant and the gaseous reactant more efficient. A higher efficiency can make it possible to operate the process at higher throughput. A further advantage of better contact between the gaseous and the liquid reactant can be reduction of the amount of by-products formed. By-product formation can be caused by heating liquid reactant in the absence of sufficient gaseous reactant. Less by-product will generally give an increase in the amount of desired product.

A process in which liquid reactant is contacted with gaseous reactant is the reaction of liquid organic compounds such as ethylbenzene or cumene with oxygen in order to obtain the corresponding hydroperoxide. Ethylbenzene hydroperoxide is applied commercially for converting propene into propylene oxide. The 1-phenylethanol which is formed thereby can subsequently be dehydrated to obtain styrene. Cumene hydroperoxide is applied commercially for preparing phenol and acetone. Alternatively, cumene hydroperoxide can be reacted with propene to obtain propylene oxide in a process similar to the process in which ethylbenzene is used. The main difference between the cumene based process and the ethylbenzene based process resides in the fact that the alcohol derived from cumene hydroperoxide which is formed upon reaction of cumene hydroperoxide and propene, generally is hydrogenated back to cumene.

It has now been found that the performance of a horizontal reactor vessel for contacting liquid reactant with gaseous reactant can be improved greatly in an easy and simple way.

### SUMMARY OF THE INVENTION

The present invention relates to a horizontal reactor vessel having a lower part and two opposite ends, which reactor vessel comprises a liquid inlet at one end, a fluid outlet at the opposite end and a gas inlet device arranged in the lower part, which reactor vessel contains at least one substantially vertical baffle-plate arranged in the direction of liquid flow through the reactor vessel during normal operation.

The present invention further relates to a process of contacting a liquid reactant with a gaseous reactant which process is carried out in a horizontal reactor vessel having a lower part and two opposite ends, which process comprises adding the liquid reactant to the reactor vessel via a liquid inlet at one end of the reactor vessel, adding the gaseous reactant via a gas inlet device arranged in the lower part at the same end and removing reaction product via a fluid outlet at the opposite end, which process is carried out in a reactor vessel further containing at least one substantially vertical baffle-plate arranged in the direction of liquid flow through the reactor vessel during normal operation.

The process is especially suitable for manufacturing hydroperoxide by contacting a liquid organic compound with an oxygen containing gas.

It was found that the present invention is especially suitable for use in large reactor vessels as applied in commercial operation. It tends to be more difficult to contact reactants efficiently in such large volume operation than in small volume commercial operation or laboratory set-ups.

### FIGURES

The invention will be illustrated by way of example in more detail with reference to the accompanying drawings, wherein

FIG. 1 shows schematically a longitudinal section of the horizontal reactor vessel;

FIG. 2 shows schematically a cross-section along the line II-II of FIG. 1;

FIG. 3 shows an alternative of the embodiment as shown in FIG. 2; and

FIG. 4 shows the cross-section of a conventional reactor vessel set-up.

### DETAILED DESCRIPTION OF THE INVENTION

The reactor vessel of the present invention is a substantially horizontal reactor. By substantially horizontal is understood substantially parallel to the plane of the horizon. Preferably, the reactor vessel for use in the present invention is tubular. Such a tubular reactor vessel can have a wide variety of shapes. For example, such a tubular reactor vessel can have a square, rectangular, circular or elliptical cross-section. For practical purposes a reactor vessel with a circular cross-section is preferred.

In a horizontal reactor the majority of the fluid flows in horizontal direction during normal operation. Horizontal reactor vessels make it possible to apply long residence times and to contact the liquid with a relatively large amount of gas. This is advantageous in the manufacture of organic hydroperoxide in view of the relatively low reaction rate.

The reactor vessel has a lower part and two opposite ends, and comprises a liquid inlet at one end and a fluid outlet at the opposite end. The reactor vessel contains at least one substantially vertical baffle-plate arranged in the direction of liquid flow through the reactor vessel during normal operation. By a substantially vertical baffle-plate is understood a baffle-plate which is situated substantially perpendicular to the plane of the horizon. As the direction of liquid flow through the reactor vessel during normal operation is from the one end to the opposite end of the reactor vessel, the baffle plate is understood to be arranged in the direction from the one end to the opposite end of the reactor vessel. The baffle-plates for use in the present invention are preferably positioned such that they are parallel to the direction in which the liquid flows during normal operation. In a horizontal tubular reactor vessel, the baffle-plates are preferably positioned substantially longitudinal.

Preferably the baffle-plate is arranged in a vertical plane parallel to or co-incident with the central longitudinal axis of the horizontal reaction vessel. For liquid mixing purposes the baffle-plates can be partly perforated.

The height of the baffle-plates can vary widely. Generally, the baffle-plates will be of from 5 to 60% of the height of the reactor vessel, more specifically of from 5 to 50%. If a single baffle-plate is present, this one can be even more than 60% of the height of the reactor vessel. The height of the horizontal reactor vessel may vary widely and for practical purposes may often range from about 0.5 to about 15 meters, preferably from about 2 to about 8 meters. Preferred heights for the baffle-plates for practical purposes may range from about 0.025 to about 9 meters, more preferably from about 0.1 to about 5 meters. Both a relatively low baffle plate (e.g. in the range from 5 to 20% of the height of the reactor vessel) and a relatively high baffle plate (e.g. in the range from 20 to 50% of the height of the reactor vessel) have been found to give the desired improved contact between gaseous reactant and liquid reactant. Very high baffle-plates (e.g. in the range from 60 to 100%, preferably 60 to 80% of the height of the reactor vessel) can also be advantageous, provided a sufficiently homogeneous reactor temperature can still be maintained. In order to maintain a sufficiently homogeneous reactor temperature it may be advantageous to use at least partly perforated baffle-plates. Someone skilled in the art will appreciate that the preferred height for a baffle-plate and the preferred extent of perforation in a given vessel depends on further circumstances such as the position of the heat exchange means and the position of the further internals.

It was found the presence of 2 or more baffle plates is especially advantageous. Therefore, it is preferred to apply 2 or more parallel baffle plates. Preferably, the number of vertical baffle-plates is of from 2 to 10, more preferably of from 2 to 5, more preferably of from 2 to 4, more preferably 2 or 3, most preferably 3.

If an odd number of baffle-plates is present, the central baffle-plate generally will be in the middle of the reactor vessel. In such case, the baffle-plate can also function as a slosh baffle to reduce the risk of sloshing in the vessel.

The baffle-plates can be connected to the wall of the reactor vessel in any way known to be suitable to someone skilled in the art, directly or indirectly. Preferably, the baffle-plates are connected directly or indirectly to the bottom of the vessel. It is preferred that the lower parts of the baffle-plates are provided with passages. To enable sufficient draining of the reactor, the distance between the wall of the reactor and the baffle-plates is preferably at least 5 mm.

The baffle-plates are substantially vertical in the present invention. The exact position of the baffle-plates depends on further circumstances. It can be preferred that the baffle-plates are situated perpendicular to the wall of the reactor vessel.

The preferred position of the baffle-plates in the reactor vessel depends on further features such as the shape of the reactor vessel, the position of the inlets and outlets and the space velocity of the fluids used. If more than one baffle-plate is present, it is preferred that these baffle-plates are distributed evenly around the centre of the vessel.

A set-up of the baffle-plates which was found to give especially good results was one in which at least 3 parallel baffle-plates were present arranged at even intervals. Even intervals means that the baffle plates are spaced apart in the lower part of the reactor such that the distances between neighbouring baffle-plates are similar.

The reactor vessel comprises a liquid inlet, one or more gas inlets and a fluid outlet. The liquid inlet and fluid outlet are placed at opposite ends of the reactor vessel in order to make maximum use of the vessel.

The reactor vessel further comprises a gas inlet device arranged in the lower part of the reactor vessel. By the lower part of the reactor vessel is understood that part of the reactor vessel lying below the horizontal plane through the central longitudinal axis of the horizontal reactor vessel.

The gas inlet device can be any gas inlet known to be suitable to someone skilled in the art. The reactor vessel according to the present invention contains at least 1 gas inlet for each reactor vessel, preferably at least 5 gas inlets. A gas inlet is considered to be an opening between the gas supply and the reactor vessel. A preferred gas inlet device is a horizontal perforated pipe extending into the lower part of the reactor vessel. The perforations of the perforated pipe open into the reactor vessel. The gas inlet most preferably used in the present invention is a so-called sparger tube.

The gas inlet device is arranged in the lower part of the reactor vessel. Preferably, the gas inlet device is near the bottom of the vessel.

A preferred gas inlet device for use in the present invention comprises at least one perforated pipe on each side of each baffle-plate. A reactor vessel containing 2 baffle-plates preferably comprises at least 3 perforated pipes. A reactor vessel containing 3 baffle-plates preferably comprises at least 4 perforated pipes.

As described herein further, a single reactor vessel can comprise several reaction zones. If this is the case, it is preferred that each reaction zone contains a gas inlet device. Preferably, each gas inlet device can be operated independently in such case.

The reactor vessel according to the present invention is especially suitable for contacting a liquid reactant and a gaseous reactant. Therefore, the present invention further relates to a process of contacting a liquid reactant with a gaseous reactant which process is carried out in a horizontal reactor vessel having a lower part and two opposite ends, which process comprises adding the liquid reactant to the reactor vessel via a liquid inlet at one end of the reactor vessel, adding the gaseous reactant via a gas inlet device arranged in the lower part and removing reaction product via a fluid outlet at the opposite end, which process is carried out in a reactor vessel further containing at least one substantially vertical baffle-plate arranged in the direction of liquid flow through the reactor vessel during normal operation.

Reaction product is removed via a fluid outlet situated opposite the liquid inlet. Additionally, one or more gas outlets can be present. The gas outlet can be present at any place in the longitudinal direction of the reactor vessel such as near the liquid inlet or near the fluid outlet.

The reactor vessel according to the present invention often will contain a heat exchange means for controlling the temperature of the reaction mixture. Such heat exchange means are preferably arranged at a position higher than the gas inlets.

The reactor vessel according to the present invention is especially suitable for the manufacture of hydroperoxide by contacting a liquid organic compound with an oxygen containing gas. Additionally, solvent can be present in such process.

The oxygen containing gas can be oxygen only or any gas in which oxygen is present in a substantial amount. Preferably, the oxygen containing gas used in the present invention is air. In such case, the excess gas which can be removed via optional gas outlet 20 will contain inert gas and a limited amount of unconverted oxygen.

The organic compound for use in the present invention can be any compound known to be suitable. An organic compound which is preferably used is ethylbenzene or cumene. Most preferably, ethylbenzene is used.

The process conditions to be used in the present invention are well known. Preferably, the temperature is of from 50 to 250° C., more preferably of from 100 to 200° C., more specifically of from 120 to 180° C. If the reactor is used in a process for the manufacture of hydroperoxide, the vessel will generally contain heat exchange means arranged in the reactor vessel to heat the reaction mixture at the start of operation and to cool when the reaction has progressed sufficiently.

The amount of oxygen containing gas to be added and the amount of organic compound to be added depends on the specific circumstances of the process such as the volume and shape of the reactor vessel and the desired concentration of hydroperoxide in the product obtained.

The pressure of the present process is not critical and can be chosen such as to best accommodate specific circumstances. Generally, the pressure near the top of the vessel will be of from atmospheric to 10×10⁵ N/m², more specifically of from 1 to 5×10⁵ N/m².

The gas removed via the gas outlet 20 can contain a considerable amount of unconverted organic compound. The exact amount of unconverted organic compound depends on the compound used and the process conditions applied. If desirable, the temperature of the gas can be lowered in order to obtain liquid unconverted organic compound. Such unconverted liquid can be recycled for further use in the process of the present invention.

The reactor vessels according to the present invention can be placed in series with further reactor vessels. In this specific set-up, the total reactor contains at least 2 reactor vessels of which one or more reactor vessels are according to the present invention and wherein the fluid outlet of one vessel is connected to the liquid inlet of a subsequent vessel. In view of the benefits of the reactor vessels according to the present invention, it is preferred that such reactor includes at least two reactor vessels according to the present invention arranged in series.

Each reactor vessel can contain one or more separate reaction zones (sometimes also referred to as separate compartments). The reaction zones can differ from each other in various aspects such as the degree of conversion which has taken place. The separate reaction zones can be created in a single reactor vessel by means which are known to someone skilled in the art. A very well known means is a vertical plate between the reaction zones perpendicular to the direction of flow which means has an opening which permits fluid to flow from one reaction zone to the subsequent reaction zone. A detailed set-up of a single reactor vessel containing a plurality of reaction zones has been described in U.S. Pat. No. 4,269,805. Such reactor vessel can be used in the present invention.

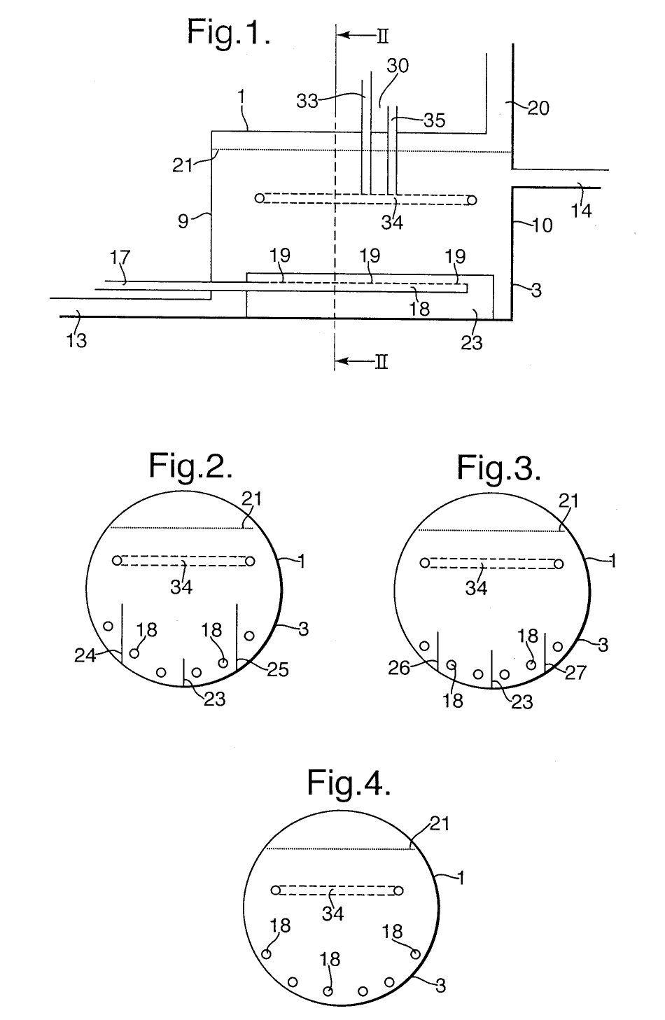

Reference is now made to FIGS. 1 and 2 showing a horizontal reactor vessel 1, which reactor vessel 1 has a lower part 3 and two opposite ends 9 and 10.

The reactor vessel 1 is provided with a liquid inlet 13 at the end 9 and a fluid outlet 14 at the opposite end 10. The lower part 3 of the reactor vessel 1 contains a gas inlet device 17. The gas inlet device 17 as shown in FIG. 1 includes a perforated pipe 18 of which the perforations 19 open into the reactor vessel 1. For the sake of clarity not all perforations have been referred to by means of a reference numeral. Dependent on the exact circumstances, it can be advantageous to remove excess gas via a separate gas outlet 20 during normal operation. This gas outlet can be absent dependent on further features of the reactor vessel and the process in which it is applied. One or more gas outlets can be present.

The fluid outlet has been depicted at the bottom of the vessel and the optional gas outlet at the top of the vessel. However, this is not required. The preferred height at which each outlet is situated depends on further circumstances as will be appreciated by someone skilled in the art. One of these circumstance is the level which the liquid generally reaches.

The reactor vessel 1 further contains at least one substantially vertical baffle-plate 23. FIGS. 2 and 3 show additional vertical baffle-plates 24 and 25, and baffle-plates 26 and 27 respectively. Baffle-plates 23, 24 and 25, and baffle-plates 23, 26 and 27 are arranged in the lower part 3 of the reactor vessel 1 and are parallel to each other. The baffle-plates 23, 24 and 25 and the baffle-plates 23, 26 and 27 are directed in the direction of liquid flow through the reactor vessel 1 during normal operation.

The reactor vessel 1 further contains heat exchange means 30 arranged therein to either heat or cool during normal operation the fluid in the reactor vessel 1. The heat exchange means 30 has an inlet (not shown) to which a supply conduit 33 is connected and an outlet (not shown) to which a discharge conduit 35 is connected. Both the supply conduit 33 and the discharge conduit 35 are connected to coil 34. Coil 34 mainly is above and below the plane depicted in the Figures. This has been indicated by dotted lines.

Reactor vessel 1 will usually substantially be filled with fluid during normal operation. A liquid level which can be encountered during normal operation has been shown by dotted line 21. The liquid level is taken to be either a level which is reached by liquid only or a level which is reached by a combination of liquid and gas.

During operation, cooling medium or heating medium can be added to heat exchange means 30 via supply conduit 33. The cooling or heating medium which has been used can be removed via discharge conduit 35. Although only a single coil 34 has been depicted, the heat exchange means will usually contain several coils.

Several heat exchange means can be present in a single reactor vessel. If a reactor vessel comprises several reaction zones, as described above, it is preferred that each reaction zone contains heat exchange means which can be operated independently.

The present invention is illustrated further in the following examples.

### EXAMPLE 1

A reactor vessel was used as depicted in FIGS. 1 and 2. The vessel had a diameter of about 5 meters and a length of about 20 meters. Ethylbenzene containing 8% wt of ethylbenzenehydroperoxide was added to this reactor vessel via inlet 13 at a rate of 660 tons/hour and air was added via gas inlet device 17 and perforated pipe 18 at a rate of 20 tons/hour. The reaction mixture was heated to a temperature of 152° C. with the help of heat exchange means 30. Upon reaching this temperature, the heat exchange means subsequently was used for cooling to remove heat produced by the exothermic reaction. The pressure in the top of the vessel was about 4×10⁵ N/m².

Gas was removed via gas outlet 20 and cooled to room temperature. The latter makes that compounds such as ethylbenzene, ethylbenzenehydroperoxide and water become liquid. It was calculated that the amount of oxygen in the remaining gas would be about 5% by mole.

### EXAMPLE 2

The process according to Example 1 was repeated in a reactor vessel as depicted in FIG. 3. Further process features were kept the same.

It was calculated that the amount of oxygen in the remaining gas would be about 6% by mole.

### EXAMPLE 3

### Comparative

The process according to Example 1 was repeated in a reactor vessel as depicted in FIG. 4. Further process features were kept the same.

It was calculated that the amount of oxygen in the remaining gas would be about 8% by mole.

A lower amount of oxygen in the gas removed from the process indicates that better use has been made of the oxygen which was added to the reaction mixture.

Therefore, the reactor vessels used in Examples 1 and 2 give a substantial improvement in process performance compared with the conventional set-up of Example 3.

1. A substantially horizontal tubular reactor vessel having a lower part and two opposite ends, which reactor vessel comprises a liquid inlet at one end, a fluid outlet at the opposite end and a gas inlet device arranged in the lower part, which reactor vessel contains at least one substantially vertical baffle-plate which is positioned substantially longitudinal in the direction from the one end to the opposite end of the reactor vessel.

2. A substantially horizontal reactor vessel according to claim 1, wherein at least one substantially vertical baffle-plate is arranged in a substantially vertical plane parallel to or co-incident with the central longitudinal axis of the horizontal reaction vessel

3. A substantially horizontal reactor vessel according to claim 1, which reactor vessel contains at least 2 parallel baffle-plates.

4. The reactor vessel according to claim 3, which vessel contains 3 baffle-plates.

5. The reactor vessel according to claim 4, which vessel contains at least 3 parallel baffle-plates arranged at even intervals.

6. The reactor vessel according to claim 1, wherein the gas inlet device includes a horizontal perforated pipe extending into the lower part of the reactor vessel.

7. The reactor vessel according to claim 6, wherein the gas inlet device includes at least one perforated pipe on each side of each baffle-plate.

8. The reactor vessel according to claim 1, wherein the lower parts of the baffle-plates is provided with passages.

9. The reactor vessel according to claim 1, which vessel further contains heat exchange means arranged in the reactor vessel.

10. A reactor that includes at least 2 reactor vessels arranged in series in which at least one of the reactor vessels is according to claim 1.

11. A process of contacting a liquid reactant with a gaseous reactant which process is carried out in a horizontal reactor vessel having a lower part and two opposite ends, which process comprises adding the liquid reactant to the reactor vessel via a liquid inlet at one end of the reactor vessel, adding the gaseous reactant via a gas inlet device arranged in the lower part and removing reaction product via a fluid outlet at the opposite end, which process is carried out in a reactor vessel further containing at least one substantially vertical baffle-plate arranged in the direction from the one end to the opposite end of the reactor vessel, wherein the substantially vertical baffle-plate is a baffle-plate which is situated substantially perpendicular to the plane of the horizon.

12. A process of manufacturing an organic hydroperoxide which process comprises contacting a liquid organic compound with an oxygen containing gas in a process according to claim 11.

13. The process according to claim 12, in which process the organic compound is cumene and/or ethylbenzene.

14. The process according to claim 12, which process is carried out at a temperature of from 100 to 200° C. and a pressure of up to 20×10⁵ N/m².
